(19) United States
(12) Patent Application Publication
(10) Pub. No.: US 2003/0104012 A1
(43) Pub. Date: Jun. 5, 2003

(54) Vaccines for the treatment of autoimmune disease

(75) Inventors: Nannette Solvason, Palo Alto, CA (US); Simonetta Mocci, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation

(21) Appl. No.: 10/146,638

(22) Filed: May 13, 2002

Related U.S. Application Data
(60) Provisional application No. 60/290,498, filed on May 11, 2001.

(51) Int. Cl. A61K 39/00

(52) U.S. Cl. 424/274.1

(57) ABSTRACT

The present invention provides pharmaceutical compositions comprising M. vaccae cells for treatment of autoimmune diseases such as diabetes, multiple sclerosis, rheumatoid arthritis, and scleroderma. The compositions may comprise either killed cells or delipidated and deglycolipidated cells.

### CROSS REFERENCE TO RELATED APPLICATIONS

This application claims priority to U.S. Ser. No. 06/290,498, filed May 11, 2001 and is related to U.S. Ser. No. 60/147,626, filed Aug. 6, 1999, and U.S. Ser. No. 09/632,893, filed Aug. 7, 2000, all of which are incorporated herein by reference.

### BACKGROUND OF THE INVENTION

The present invention relates to compositions and methods for treating autoimmune diseases. The compositions comprise as an active ingredient Mycobacteria vaccae.

A number of pathological responses involving unwanted immune responses are known. For instance, autoimmune disease is a particularly important class of deleterious immune response. In autoimmune diseases, self-tolerance is lost and the immune system attacks “self” tissue as if it were a foreign target. More than 30 autoimmune diseases are presently known, including rheumatoid arthritis (RA), insulin-dependent diabetes mellitus (IDDM), multiple sclerosis (MS), myasthenia gravis (MG), systemic lupus erythematosis (SLE), and scleroderma.

Although many advances have been made in the treatment of autoimmune disease, new therapeutic approaches to treatment and prevention of these diseases are needed. The present invention addresses these and other needs.

### SUMMARY OF THE INVENTION

The present invention provides pharmaceutical compositions comprising killed M. vaccae cells or P-VAC for treatment of autoimmune diseases. P-VAC is a delipidated and deglycolipidated fraction derived from M. vaccae cells. The compositions of the invention may further comprise an adjuvant. The compositions may be administered according to standard methods well known in the art. Typically, the compositions are administered parenterally, for example subcutaneously or intraperitonealy. The methods of the invention can be used to treat autoimmune diseases such as diabetes, multiple sclerosis, rheumatoid arthritis, and scleroderma.

In some embodiments, the methods of the invention are used to treat scleroderma. In these embodiments, the compositions of the invention may comprise an immunostimulant. The immunostimulant can be an adjuvant selected from the group consisting of mineral oil, Bortadella pertussis, Freund's Incomplete Adjuvant, Freund's Complete Adjuvant, CWS, TDM, Leif, an aluminum salt, GM-CSF, interleukin-2, interleukin-7, interleukin-12, Montanide ISA 720, SAF, ISCOMS, MF-59, an SBAS adjuvant, and Detox. Alternatively, the immunostimulant is an adjuvant selected from the group consisting of AS-2 or derivative thereof, a biodegradable microsphere, monophosphoryl lipid A (MPL), 3-de-O-acylated monophosphoryl lipid A (3D-MPL), Quil A, CpG, a saponin or mimetic thereof, and an aminoalkyl glucosaminide 4-phosphate.

In the methods, the P-VAC can be administered parenterally, subcutaneously, or intraperitoneally.

### BRIEF DESCRIPTION OF THE DRAWINGS

FIG. 1 demonstrates a statistically significant reduction in the incidence of diabetes following multiple doses of M-VAC.

FIG. 2 demonstrates a statistically significant reduction in the incidence of diabetes following multiple doses of PVAC/IFA.

FIG. 3 demonstrates a statistically significant reduction in the incidence of diabetes following either pre-cyclo OR post-cyclo doses of PVAC/IFA.

FIG. 4 demonstrates a statistically significant reduction in the incidence of diabetes following a single dose of PVAC/IFA on day 4 post-cyclophosphamide treatment.

FIG. 5 shows the protocol and results for experiments demonstrating the ability of MVAC to lower the incidence of EAE.

FIG. 6 shows the protocol for experiments that test the ability of multiple pre- and post-disease induction injections of PVAC/IFA to block symptoms of EAE.

FIG. 7 shows the results for experiments demonstrating that multiple pre- and post-disease induction injections of PVAC/IFA lower the incidence of EAE.

FIG. 8 shows the protocol for experiments that test the ability of two post-disease induction injections of PVAC/IFA to block symptoms of EAE.

FIG. 9 shows the results of experiments demonstrating that two injections of PVAC/IFA given post-disease induction lowers the symptoms of EAE.

FIG. 10 shows the results of experiments demonstrating the ability of PVAC/IFA to act as adjuvants in the induction of EAE in the SJL mouse.

FIG. 11 shows the scoring guide used in the evaluation of CIA model.

FIG. 12 shows the protocol for experiments testing the ability of PVAC to lower the incidence of arthritis in the collagen induced arthritis (CIA) model.

FIG. 13 shows the results suggesting that PVAC/IFA lowers the severity of arthritis in CIA model.

### DESCRIPTION OF THE SPECIFIC EMBODIMENTS

The present invention provides pharmaceutical compositions comprising antigenic material derived from Mycobacterium vaccae. The compositions are generally useful in the treatment of pathological conditions such as autoimmune diseases.

The therapeutic compositions of the invention may comprise dead cells of M. vaccae, referred to here as M-VAC. The means by which the cells have been killed is not critical and may be done by e.g. autoclaving or irradiation. Preparation of pharmaceutical compositions comprising such cells are described for example in EP 630 259, and U.S. Pat. No. 5,833,996. A strain of M. vaccae denoted R877R has been deposited under the Budapest Convention at the National Collection of Type Cultures (NCTC) under the number NCTC 11659. (see, Stanford and Paul, Ann. Soc. Belge Med, Trop. 53:141-389 (1973)).

Alternatively, the compositions of the invention may comprise delipidated and deglycolipidated fraction derived from M. vaccae cells (DD-M.V.) referred to here as P-VAC. Preparation of such cells is described for instance in WO 99/32634 and WO 98/8542 (see Example 1 for details).

The M. vaccae cells or modified cells can be formulated in pharmaceutical compositions useful for administration to mammals, particularly humans, to treat and/or prevent deleterious autoimmune responses. Suitable formulations are found in Remington's Pharmaceutical Sciences, Mack Publishing Company, Philadelphia, Pa., 17th ed. (1985).

Pharmaceutical Compositions

The present invention concerns formulation of the M. vaccae compositions disclosed herein in pharmaceutically-acceptable solutions for administration to an animal, either alone, or in combination with one or more other modalities of therapy.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tablets, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

2. Injectable Delivery

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified. ps 3. Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

4. Liposome-,Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the compositions disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trials examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977, 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution. However, a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for hours or days, depending on their composition, and half lives in the blood range from minutes to several hours. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominant site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

Vaccines

In certain preferred embodiments of the present invention, vaccines are provided. The vaccines will generally comprise one or more pharmaceutical compositions, such as those discussed above, in combination with an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g. Fullerton, U.S. Pat. No. 4,235,877). Vaccine preparation is generally described in, for example, Powell & Newman, eds., Vaccine Design (the subunit and adjuvant approach) (1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte response in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium species or Mycobacterium derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996).

Another preferred adjuvant is a saponin or saponin mimetics or derivatives, preferably QS21 (Aquila Biopharmaceuticals Inc., Framingham, Ma.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438 (1996)) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLE 1

### Preparation of Delipidated and Deglycolipidated M. Vaccae (P-VAC)

P-VAC was prepared as generally described in WO 99/32634. A summary of the protocol follows.

Heat-killed M. vaccae was prepared using standard methods. To prepare delipidated M. vaccae, the autoclaved M. vaccae was pelleted by centrifugation, the pellet washed with water, collected again by centrifugation and then freeze-dried. An aliquot of this freeze-dried M. vaccae was set aside and referred to as lyophilised M. vaccae. When used in experiments it was resuspended in PBS to the desired concentration. Freeze-dried M. vaccae was treated with chloroform/methanol (2:1) for 60 mins at room temperature to extract lipids, and the extraction was repeated once. The delipidated residue from chloroform/methanol extraction was further treated with 50% ethanol to remove glycolipids by refluxing for two hours. The 50% ethanol extraction was repeated two times. The residue from the 50% ethanol extraction was freeze-dried and weighed. The delipidated and deglycolipidated M. vaccae (DD-M. vaccae) was resuspended in phosphate-buffered saline by sonication, thus producing P-VAC. This preparation in preferred embodiments is used for treatment of autoimmune diseases without further processing. In some embodiments, the preparation is autoclaved before administration.

### EXAMPLE 2

### M-Vac can Ameliorate Disease in a Nod Model for Autoimmune IDDM

This example demonstrates the use of killed M. vaccae cells (M-VAC) to ameliorate disease in NOD mice, a murine model for autoimmune IDDM. The disease in these animals is characterized by anti-islet cell antibodies, severe insulitis, and evidence for autoimmune destruction of the β-cells. Seventy to ninety percent of female and 20-30% of male animals spontaneously develop diabetes within the first six months of life. The disease can also be induced in these mice by administration of cyclophosphamide.

Protocol and Results for M-VAC experiment

1. Normorglycaemic NOD male mice (age 6-8 weeks) were divided into 3 experimental groups

2. Group 1 received 500 μg of MVAC IP

3. Group 2 received 500 μg of MVAC IP on day (−2), 1, 4, 7 and 14. Cyclophosphamide was given on Day 0.

4. Group 3 received cyclophosphamide injections only on Day 0

5. Mice were monitored for development of diabetes by DIASTIX on day 7 and every Monday, Wednesday and Friday thereafter until day 21 when the experiment was terminated.

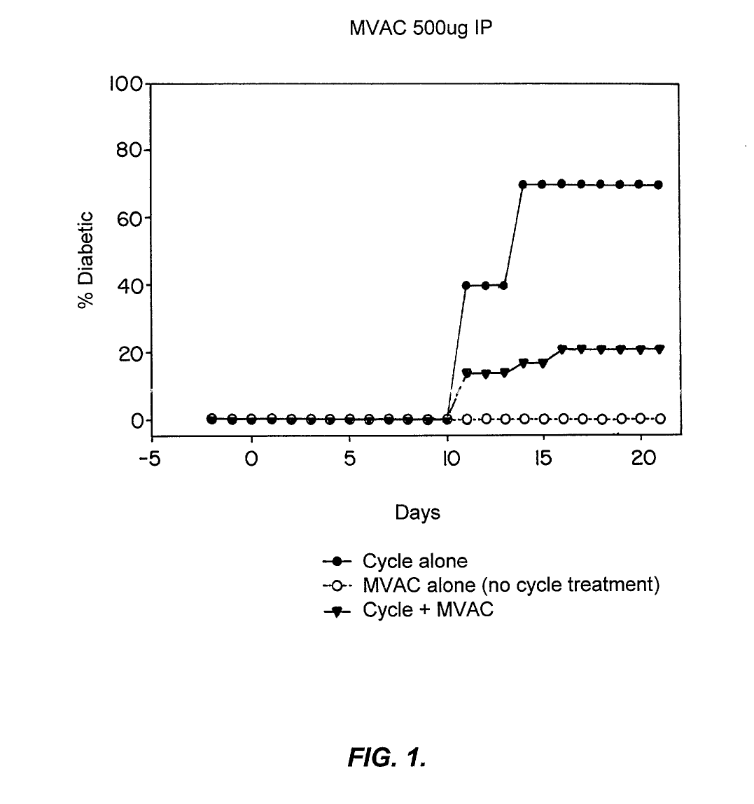

As can be seen in FIG. 1, multiple injections of MVAC given by IP injection provides statistically significant protection from the development of diabetes in this model (p=0.0001). The administration of MVAC alone (in the absence of cyclophosphamide) had no effect on the mice.

### EXAMPLE 3

### P-VAC Alone cannot Ameliorate Disease in a Nod Model for Autoimmune IDDM

This example evaluates the use of delipidated, deglycolipidated M. vaccae (P-VAC) to ameliorate disease in NOD mice, a murine model for autoimmune IDDM. The disease in these animals is characterized by anti-islet cell antibodies, severe insulitis, and evidence for autoimmune destruction of the β-cells. Seventy to ninety percent of female and 20-30% of male animals spontaneously develop diabetes within the first six months of life. The disease can also be induced in these mice by administration of cyclophosphamide with between 60-80% of treated mice developing diabetes.

General Protocol

Normorglycaemic NOD (non-obese diabetic) male mice between the ages of 8 and 10 weeks were used in this model system. Cyclophosphamide was administered at a single dose of 250 mg/kg body weight by intraperitoneal injection; Day 0 is the date of cyclophosphamide injection. P-VAC suspended in 100 μl PBS was administered at varying doses, via intraperitoneal (IP) or subcutaneous (SC) injection, and both pre- and post-induction. Disease progression was monitored at Days 7, 9, 11, 14, 16, and 21 by tests for glycosuria (DIASTIX).

A common analytic strategy was used each of the four experiments. First, each of the experimental groups was compared to the control group with respect to the number of non-diabetic (i.e., non-glycosuric) mice observed on the final experimental day (i.e., Day 21, following cyclophosphamide injection). For each comparison, this results in a 2×2 table of counts (non-glycosuric vs. glycosuric status, by experimental vs. control group); the proportions of non-glycosuric mice in the two groups were compared using Pearson's chi-square test for equality of proportions.

Second, survival analysis methods were used to look for differences between experimental groups and the control group in the time course of the development of hyperglycemia. Specifically, the Kaplan-Meier estimate of the survivor function was computed for each experimental group, where “survivor” means (in this case) a mouse not yet glycosuric. That is, the survivor curve is plot of the estimated chance of a mouse remaining non-glycosuric, evaluated at each of the seven experimental days following cyclophosphamide injection. The log-rank test was then used to test for equality of survivor functions between each of the experimental groups and the control group.

Experimental Design and Results

In the first experiment, P-VAC alone was administered IP at Day 1, 4, 7, and 14 after disease induction using different dose regimens (1.0 μg, 0.1 μg, or 0.01 μg). The results are seen in Table 1. Statistical analyses indicate that IP injection of 1.0 μg, 0.1 μg, or 0.01 μg of P-VAC after cyclophosphamide induction has no effect on the incidence of diabetes occurrence in this model system.

In a second experiment, the P-VAC dosing regimen was changed as follows:

P-VAC was administered IP both pre- and post-disease induction at Day (−5), (−2), +1, +4, +7 and +14 using different dose regimens (100 μg or 10 μg). The results are seen in Table 1. Statistical analyses indicate that IP treatment with 10 μg or 100 μg P-VAC both pre- and post-disease induction has no effect on the incidence of diabetes occurrence in this model system.

In a third experiment, the route of administration was altered. P-VAC was administered by SC injection both pre- and post-disease induction at Day (−5), (−2), +1, +4, +7 and +14 using different dose regimens (100 μg or 10 μg). The results are seen in Table 1. Statistical analyses indicate that SC treatment with 10 μg or 100 μg P-VAC both pre- and post-disease induction has no effect on the incidence of diabetes occurrence in this model system.

In a fourth experiment, the previous experiment was repeated with larger groups of animals and the first P-VAC treatment date changed to Day (−4) before disease induction with cyclophosphamide. The results are seen in Table 1. Statistical analyses indicate that SC treatment with 10 μg or 100 μg P-VAC both pre- and post-disease induction has no effect on the incidence of diabetes occurrence in this model system

For each of Experiments 1, 2, 3, and 4, no significant difference was found between any experimental group and the corresponding control group with respect to the proportion of non-glycosuric mice on Day 21 (Pearson chi-square p>0.05, in each case). Similarly, no significant difference was found between the survivor functions of any experimental group and the corresponding control group (log-rank p>0.05, in each case). While no significant differences were found in each of Experiments 2, 3, and 4, the proportion of non-glycosuric mice on Day 21 was greater in the 100 μg PVAC group than in the corresponding control group.

Table 1 Analysis of PVAC Effects in IDDM Model in NOD Mice

Table 1 summarizes the results of the statistical analyses. The first column displays labels for the various groups, the second column shows the number of mice (n) in the group, and the third column shows the number of non-glycosuric mice in that group on the final experimental day (Day 21). The fourth column displays the p-value for the Pearson chi-square test comparing the proportion of non-glycosuric mice on Day 21 vs. the corresponding proportion for the control group. Column five of each table shows the median “survival time” in each group, i.e., the median number of days until the appearance of hyperglycemia. (Note that, in some cases, fewer than half of the animals in a group became glycosuric during the experimental period. In those cases, the median time is shown as “>21”.) Finally, column six in each table shows the p-value for the log-rank test for equality of survivor functions between an experimental group and the control group.

TABLE 1
Analysis of PVAC effects in IDDM model in NOD mice

| | | | | | |
|---|---|---|---|---|---|
| | | Non-glycosuric | Chi | Median | Log |
| Experimental | Sample | Mice on | Square | Days To | Rank |
| Group | Size (n) | Day 21 | p-value | Glycosuria | p-value |

Experiment 1

| | | | | | |
|---|---|---|---|---|---|
| 1.0 μg PVAC | 16 | 5 | 0.988 | 14 | 0.786 |
| 0.1 μg PVAC | 21 | 4 | 0.34 | 11 | 0.431 |
| 0.01 μg PVAC | 17 | 4 | 0.585 | 16 | 0.565 |
| Control | 29 | 9 | | 14 | |

Experiment 2

| | | | | | |
|---|---|---|---|---|---|
| 100 μg PVAC | 20 | 7 | 0.216 | 14 | 0.417 |
| 10 μg PVAC | 15 | 2 | 0.877 | 11 | 0.933 |
| Control | 13 | 2 | | 11 | |

Experiment 3

| | | | | | |
|---|---|---|---|---|---|
| 100 μg PVAC | 20 | 11 | 0.20 | >21 | 0.325 |
| 10 μg PVAC | 20 | 13 | 0.058 | >21 | 0.139 |
| Control | 20 | 7 | | 16 | |

Experiment 4

| | | | | | |
|---|---|---|---|---|---|
| 100 μg PVAC | 24 | 14 | 0.474 | >21 | 0.552 |
| 10 μg PVAC | 25 | 12 | 0.967 | >21 | 0.974 |
| Control | 19 | 9 | | >21 | |

### EXAMPLE 4

### PVAC/IFA Administered Pre- and Post-Disease Induction Ameliorates Disease in an NOD Model for Autoimmune IDDM

This example demonstrates the use of multiple pre-and post-PVAC/IFA injections to lower the incidence of disease in NOD mice, a murine model for autoimmune IDDM. The disease in these animals is characterized by anti-islet cell antibodies, severe insulitis, and evidence for autoimmune destruction of the β-cells. Seventy to ninety percent of female and 20-30% of male animals spontaneously develop diabetes within the first six months of life. The disease can also be induced in these mice by administration of cyclophosphamide.

Protocol and Results for Experiments Designed to Evaluate the Ability of PVAC/IFA to Lower Incidence of Diabetes in an NOD Model for Autoimmune IDDM.

1. Normoglycaemic male NOD mice (age 6-8 weeks) were divided into 3 groups

2. Group 1 was given 100 μg PVAC emulsified in IFA on days (−5), (−2), 1, 4 and 7 days post cyclophosphamide administration

3. Group 2 was given PBS emulsified in IFA on days (−5), (−2), 1, 4 and 7 days post cyclophosphamide adminstration

4. Group 3 was given cyclophosphamide only on day 0.

5. Mice were monitored for the development of diabetes by DIASTIX on day 7 and every other day thereafter until day 21 when the experiment was terminated.

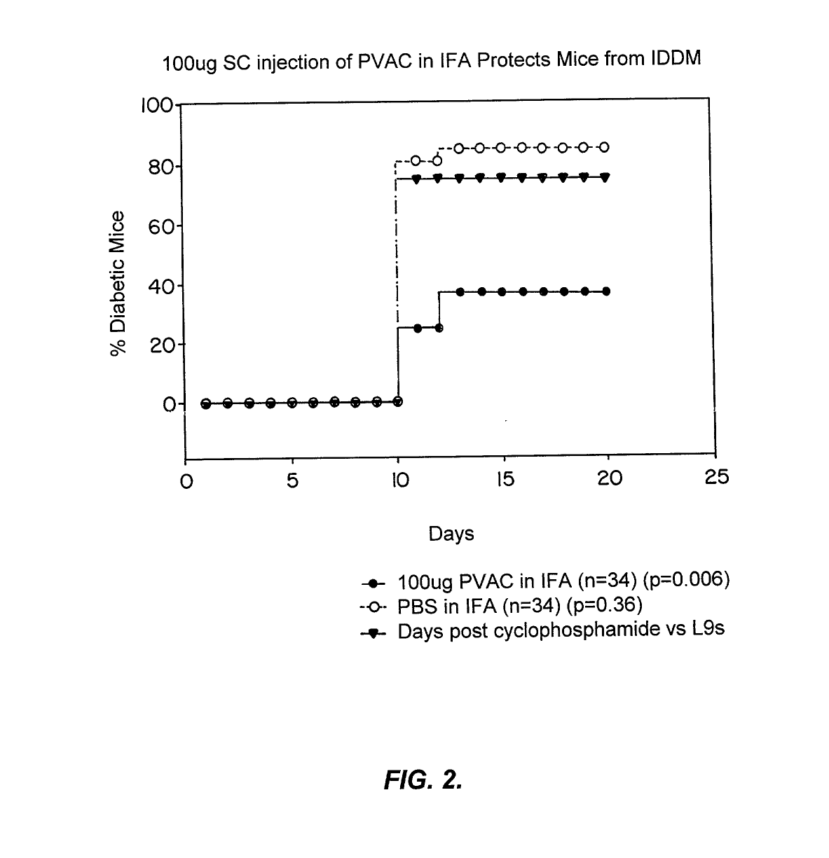

As seen in FIG. 2, a statistically significant decrease in the incidence of IDDM was evident in the mice receiving multiple injections of PVAC/IFA (p=0.006).

### EXAMPLE 5

### PVAC/IFA Administered Post-Disease Induction Ameliorates Disease in an NOD Model for Autoimmune IDDM

This example demonstrates the use of multiple post-PVAC/IFA injections to lower the incidence of disease in NOD mice, a murine model for autoimmune IDDM. The disease in these animals is characterized by anti-islet cell antibodies, severe insulitis, and evidence for autoimmune destruction of the β-cells. Seventy to ninety percent of female and 20-30% of male animals spontaneously develop diabetes within the first six months of life. The disease can also be induced in these mice by administration of cyclophosphamide.

Protocol and Results for Comparison of Pre- and Post-Cyclophosphamide Injections of PVAC Emulsified in IFA.

1. Normoglycaemic male NOD mice (age 6-8 weeks) were divided into 4 groups

2. Group 1 received injections of 100 μg PVAC/IFA on days (−5), (−2), 1, 4, 7 and 14 post cyclophosphamide injection.

3. Group 2 received injections of 100 μg PVAC/IFA on days (−5) and (−2) post cyclophosphamide injection.

4. Group 3 received injections of 100 μg PVAC/IFA on days 1, 4, 7 and 14 post cyclophosphamide injection

5. Group 4 received injections of PBS/IFA on days (−5), (−2), 1, 4, 7 and 14 post cyclophamide.

6. Mice were monitored for the development of diabetes by DIASTIX on day 7 and every other day thereafter until day 21 when the experiment was terminated.

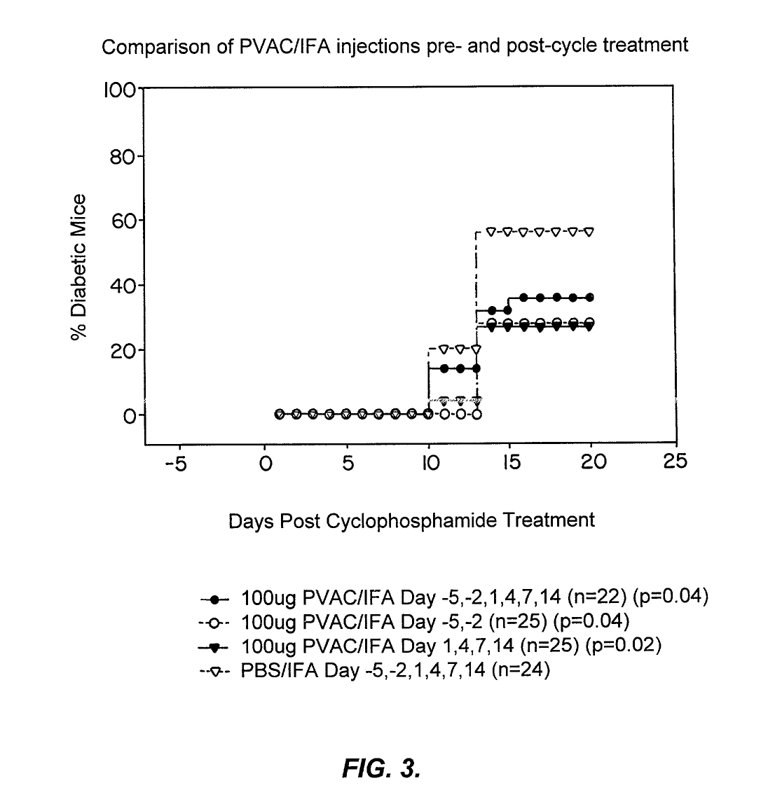

As seen in FIG. 3, injections of PVAC/IFA given pre-cyclophosphamide (p=0.04) OR post-cyclophosphamide (p=0.02) are equally as efficacious as the pre- AND post-cyclophosphamide treatment (p=0.04).

### EXAMPLE 6

### Administration of PVAC/IFA on Day 4 Post Disease Induction Ameliorates Disease in an NOD Model for Autoimmune IDDM

This example demonstrates the use of a single administration of PVAC/IFA to lower the incidence of disease in NOD mice, a murine model for autoimmune IDDM. The disease in these animals is characterized by anti-islet cell antibodies, severe insulitis, and evidence for autoimmune destruction of the β-cells. Seventy to ninety percent of female and 20-30% of male animals spontaneously develop diabetes within the first six months of life. The disease can also be induced in these mice by administration of cyclophosphamide

Protocol and Results for Determination of the Adminstration Date for PVAC/IFA which Provides Maximum Protection.

1. Normoglycaemic male NOD mice (age 6-8 weeks) were divided into 5 groups.

2. Group 1 received 100 μg PVAC/IFA on day 4 post cyclophosphamide injection

3. Group 2 received 100 μg PVAC/IFA on day 7 post cyclophosphamide injection

4. Group 3 received 100 μg PVAC/IFA on day 10 post cyclophosphamide injection

5. Group 4 received 100 μg PVAC/IFA on day 13 post cyclophosphamide injection.

6. Group 5 received cyclophosphamide alone on day 0.

7. Mice were monitored for the development of diabetes by DIASTIX on day 7 and every other day thereafter until day 21 when the experiment was terminated.

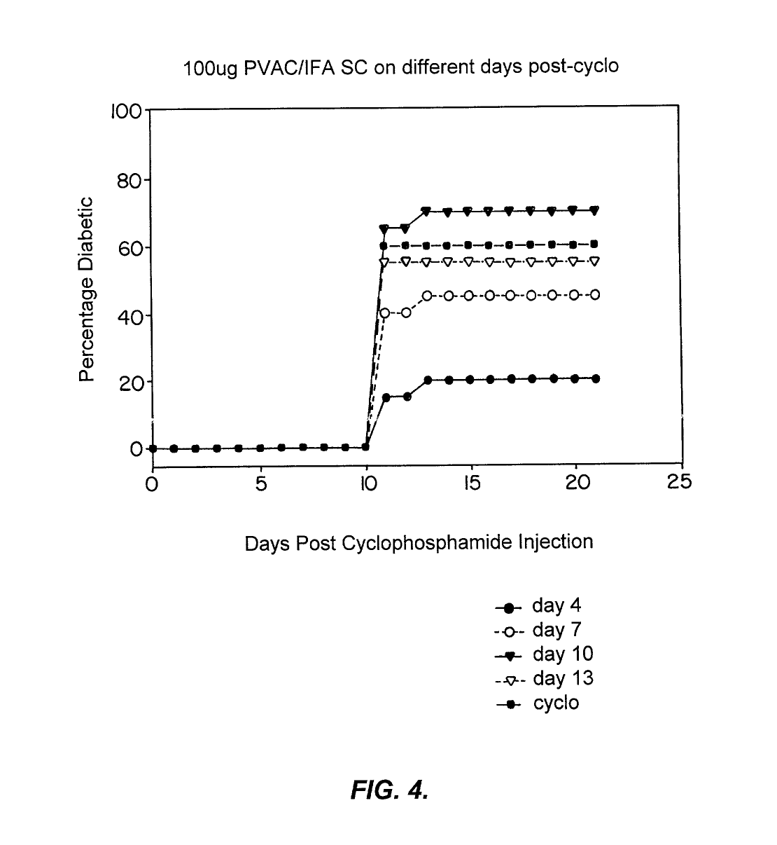

As seen in FIG. 4, administration of PVAC/IFA on day 4 provides maximum protection from the development of diabetes in this model (p=0.006).

### EXAMPLE 7

### M-VAC can Ameliorate Disease in an EAE Model for Multiple Sclerosis

This example demonstrates the use of killed M. vaccae cells (M-VAC) to ameliorate disease in experimental allergic encephalomyelitis (EAE) a model for multiple sclerosis. EAE is an autoimmune inflammatory disorder of genetically susceptible mice that is mediated by autoantigen-specific CD4+MHC class II restricted T cells. In susceptible SJL/J mice, the disease can display a relapsing-remitting clinical course of paralysis, which makes it an ideal system to study the efficacy of various immunoregulatory strategies both in the prevention and treatment of disease.

EAE can be induced by using either crude preparations of spinal cord homogenate or myelin or immunodominant encephalitogenic peptides derived from myelin, such as PLP 139-151 peptide. In this case, remitting-relapsing (RR) EAE is induced in female SJL mice (8-12 wks old) by immunization with 150 μg/mouse of the immunodominant peptide PLP 139-151. The peptide is dissolved in PBS and emulsified with an equal volume of CFA containing 600 μg of Mycobacterium tuberculosis H37Ra. Each mouse receives 0.2 ml of the emulsion, which will be administrated subcutaneously (sc) and distributed over three sites on the flank and base of tail. Mice are also given an injection of the coadjuvant pertussis toxin (200 ng/mouse intraperitoneally (ip)) on the day of immunization and 48 hrs later. Beginning approximately 9 days after immunization, animals are weighed daily and observed for the onset of neurological dysfunction. Disease is graded as follows: 0, normal; 0.5, stiff tail; 1, limp tail; 1.5, limp tail with inability to right; 2, paralysis of one limb; 2.5, paralysis of one limb and weakness of one other limb; 3, complete paralysis of both hind limbs; 4, moribund; 5, death. Mice are usually followed for up to 75-80 days after immunization for relapses.

Experimental Design and Results

Based on the data obtained with M-VAC treatment in the NOD mouse model, we decided to evaluate the effect of M-VAC on the murine EAE model.

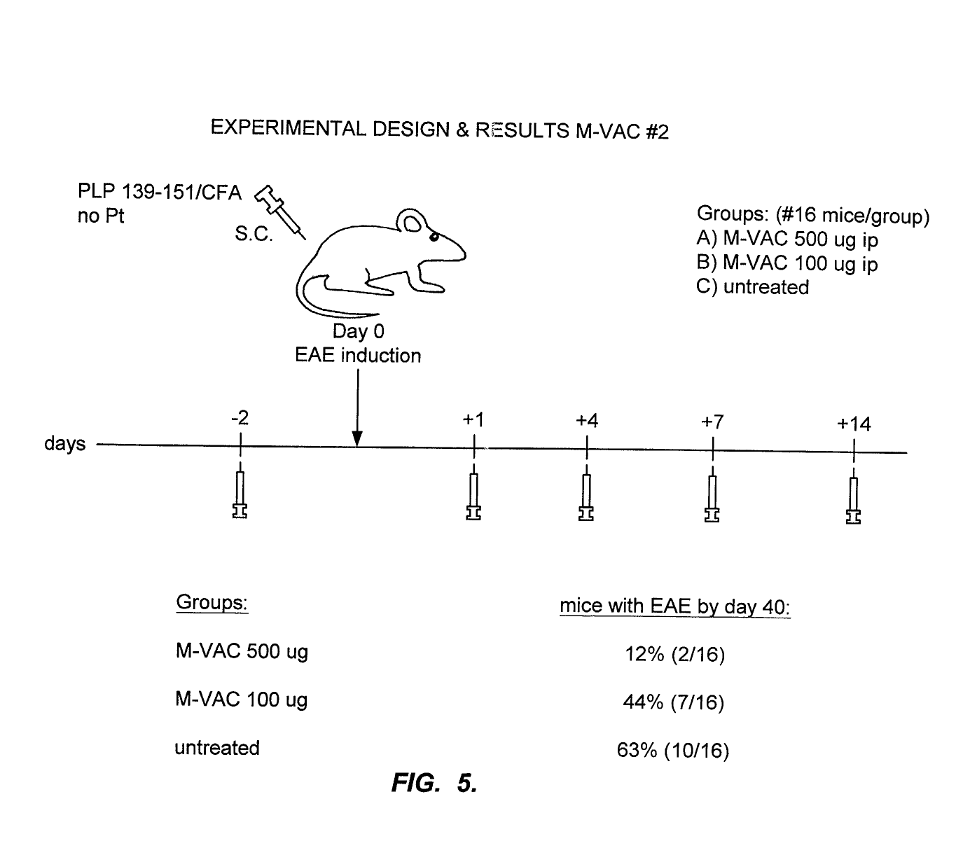

In the first experiment, EAE was induced in SJL mice according to the protocol used previously and M-VAC was given at the dose of 500 μg/mouse ip in PBS at day −2, +1, +4, +7 and +14 after disease induction. By day +14, 12 out of 16 mice treated with M-VAC were dead whereas only 1 mouse out of 15 dead in the untreated control. This result raised the question of whether M-VAC had any toxic effect in conjunction with the co-injection of pertussis toxin (delivered IP as well). Therefore we repeated the same experiment in SJL mice where EAE was induced without the co-administration of pertussis toxin. In addition, 2 different doses of M-VAC, 500 and 100 μg/mouse in PBS were used in this experiment. The results (see, FIG. 5) indicated that the toxic effect observed previously was indeed caused by the coinjection of pertussis and not by M-VAC itself (mice with no disease induction and treated with M-VAC do not show toxic effect as well, data not shown). Furthermore, mice receiving 500 μg/mouse of M-VAC showed a significant reduction of disease incidence.

### EXAMPLE 8

### PVAC Alone cannot Ameliorate Disease in an EAE Model for Multiple Sclerosis

This example demonstrates the use of delipidated, deglycolipidated M. vaccae to ameliorate disease in experimental allergic encephalomyelitis (EAE), a model for multiple sclerosis. EAE is an autoimmune inflammatory disorder of genetically susceptible mice that is mediated by autoantigen-specific CD4+MHC class II restricted T cells. In susceptible SJL/J mice, the disease can display a relapsing-remitting clinical course of paralysis, which makes it an ideal system to study the efficacy of various immunoregulatory strategies both in the prevention and treatment of disease.

General Protocol

EAE can be induced by using either crude preparations of spinal cord homogenate or myelin or immunodominant encephalitogenic peptides derived from myelin, such as PLP 139-151 peptide. In this case, remitting-relapsing (RR) EAE is induced in female SJL mice (8-12 wks old) by immunization with 200 □g/mouse of the immunodominant peptide PLP 139-151. The peptide is dissolved in PBS and emulsified with an equal volume of CFA containing 600 μg of Mycobacterium tuberculosis H37Ra. Each mouse receives 0.2 ml of the emulsion, which will be administrated subcutaneously (sc) and distributed over three sites on the flank and base of tail. Mice are also given an injection of the coadjuvant pertussis toxin (200 ng/mouse intraperitoneally (ip)) on the day of immunization and 48 hrs later. Beginning approximately 9 days after immunization, animals are weighed daily and observed for the onset of neurological dysfunction. Disease is graded as follows: 0, normal; 0.5, stiff tail; 1, limp tail; 1.5, limp tail with inability to right; 2, paralysis of one limb; 2.5, paralysis of one limb and weakness of one other limb; 3, complete paralysis of both hind limbs; 4, moribund; 5, death. Mice are usually followed for 60-70 days after immunization for relapses.

A common analytic strategy was used for these experiments. The worst disease status (highest severity grade) was calculated for each mouse. Each of the experimental groups was then compared to the control groups with respect to the worst disease status observed, in two ways. First, the groups were compared in terms of whether any disease-related symptoms were observed, i.e. worst disease status=0 vs. worst disease status>0. Second, the groups were compared in terms of whether any paralysis (or a worse symptom) was observed, i.e., worst disease status<2 vs. worst disease status≧2. Each possible comparison resulted in a 2×2 table of counts (control vs. experimental group, by two types of worst disease status). Fisher's exact test was used to test the hypothesis of no difference between groups in the proportions of mice in the two worst disease status categories.

Experimental Design and Results

In the first experiment, EAE was induced in female SJL mice (8-12 wks old) by sc injection of PLP 139-151/CFA+Pt (day 0) and P-VAC was administrated IP at day +1, +4, +7 and +14 after disease induction using different dose regimens (0.01 μg/mouse; 0.1 μg/mouse, 1 μg/mouse).

The results, expressed as the number of symptomatic and paralytic mice, showed no significant effect of any of the doses tested in the development of disease (Table 3). No statistically significant differences were found between the control group and any of the experimental groups comparing the worst disease status of each mouse during the entire experimental period.

In a second experiment, the P-VAC regimen and route of administration were changed as follows:

SJL mice received either 10 or 100 μg/mouse of P-VAC delivered ip or sc at day −5, −2, +1, +4, +7 and +14 after disease induction. The responses of the mice in this experiment were bi-phasic; consequently, the disease comparisons were performed for the time periods Days 1-31 and Days 32-62 post-disease induction. There was a statistically significant difference between the 10 μg P-VAC (ip) treatment groups and the control group during the first 31 days of the experiment; fewer mice in the P-VAC treatment groups showed signs of paralysis (p=0.048) (Table 3). These results indicate that P-VAC can protect against the development of EAE when animals are treated both before and after disease induction.

TABLE 3
Analysis of PVAC Effects in EAE Model
Experiment 1

| | | Symptomatic | Fisher's | Paralytic | Fisher's |
|---|---|---|---|---|---|
| Experimental | Sample | Mice | Exact | Mice | Exact |
| Group | Size (n) | Days 1-65 | p-value | Days 1-65 | p-value |
| 1.0 μg PVAC | 12 | 12 | * | 9 | 0.70 |
| 0.1 μg PVAC | 12 | 12 | * | 9 | 0.70 |
| 0.01 μg PVAC | 11 | 11 | * | 10 | 0.20 |
| PBS | 12 | 12 | * | 11 | 0.18 |
| Control | 15 | 15 | * | 10 | |

Experiment 2 (Phase 1)

| | | Symptomatic | Fisher's | Paralytic | Fisher's |
|---|---|---|---|---|---|
| Experimental | Sample | Mice | Exact | Mice | Exact |
| Group | Size (n) | Days 1-31 | p-value | Days 1-31 | p-value |
| 100 μg PVAC, ip | 15 | 15 | * | 15 | * |
| 10 μg PVAC, ip | 16 | 16 | * | 11 | 0.048 |
| 100 μg PVAC, sc | 16 | 16 | * | 14 | 0.488 |
| 10 μg PVAC, sc | 15 | 15 | * | 14 | 1.000 |
| 100 μl PBS, ip | 16 | 16 | * | 15 | 1.000 |
| Control | 13 | 13 | * | 13 | |

Experiment 2 (Phase 2)

| | | Symptomatic | Fisher's | Paralytic | Fisher's |
|---|---|---|---|---|---|
| Experimental | Sample | Mice | Exact | Mice Days | Exact |
| Group | Size (n) | Days 32-62 | p-value | 32-62 | p-value |
| 100 μg PVAC, ip | 15 | 15 | * | 8 | 0.276 |
| 10 μg PVAC, ip | 16 | 16 | * | 8 | 0.451 |
| 100 μg PVAC, sc | 16 | 16 | * | 6 | 1.000 |
| 10 μg PVAC, sc | 15 | 15 | * | 7 | 0.460 |
| 100 μl PBS, ip | 16 | 16 | * | 9 | 0.264 |
| Control | 13 | 13 | * | 4 | |

* No mouse in this group was symptom-free

### EXAMPLE 9

### Administration of PVAC/IFA Pre- and Post-Disease Induction can Ameliorate Disease in an EAE Model of Multiple Sclerosis

This example demonstrates the use of delipidated, deglycolipidated M. vaccae to ameliorate disease in experimental allergic encephalomyelitis (EAE), a model for multiple sclerosis. EAE is an autoimmune inflammatory disorder of genetically susceptible mice that is mediated by autoantigen-specific CD4+MHC class II restricted T cells. In susceptible SJL/J mice, the disease can display a relapsing-remitting clinical course of paralysis, which makes it an ideal system to study the efficacy of various immunoregulatory strategies both in the prevention and treatment of disease.

General Protocol

EAE can be induced by using either crude preparations of spinal cord homogenate or myelin or immunodominant encephalitogenic peptides derived from myelin, such as PLP 139-151 peptide. In this case, remitting-relapsing (RR) EAE is induced in female SJL mice (8-12 wks old) by immunization with 200 μg/mouse of the immunodominant peptide PLP 139-151. The peptide is dissolved and emulsified with an equal volume of CFA containing 600 μg of Mycobacterium tuberculosis H37Ra. Each mouse receives 0.2 ml of the emulsion, which will be administrated subcutaneously (sc) and distributed over three sites on the flank and base of tail. Mice are also given an injection of the coadjuvant pertussis toxin (200 ng/mouse intraperitoneally (ip)) on the day of immunization and 48 hrs later. Beginning approximately 9 days after immunization, animals are weighed daily and observed for the onset of neurological dysfunction. Disease is graded as follows: 0, normal; 0.5, stiff tail; 1, limp tail; 1.5, limp tail with inability to right; 2, paralysis of one limb; 2.5, paralysis of one limb and weakness of one other limb; 3, complete paralysis of both hind limbs; 4, moribund; 5, death. Mice are usually followed for 60-70 days after immunization for relapses.

A common analytic strategy was used for these experiments. The worst disease status (highest severity grade) was calculated for each mouse. Each of the experimental groups was then compared to the control groups with respect to the worst disease status observed, in two ways. First, the groups were compared in terms of whether any disease-related symptoms were observed, i.e. worst disease status=0 vs. worst disease status>0. Second, the groups were compared in terms of whether any paralysis (or a worse symptom) was observed, i.e., worst disease status<2 vs. worst disease status≧2. Each possible comparison resulted in a 2×2 table of counts (control vs. experimental group, by two types of worst disease status). Fisher's exact test was used to test the hypothesis of no difference between groups in the proportions of mice in the two worst disease status categories.

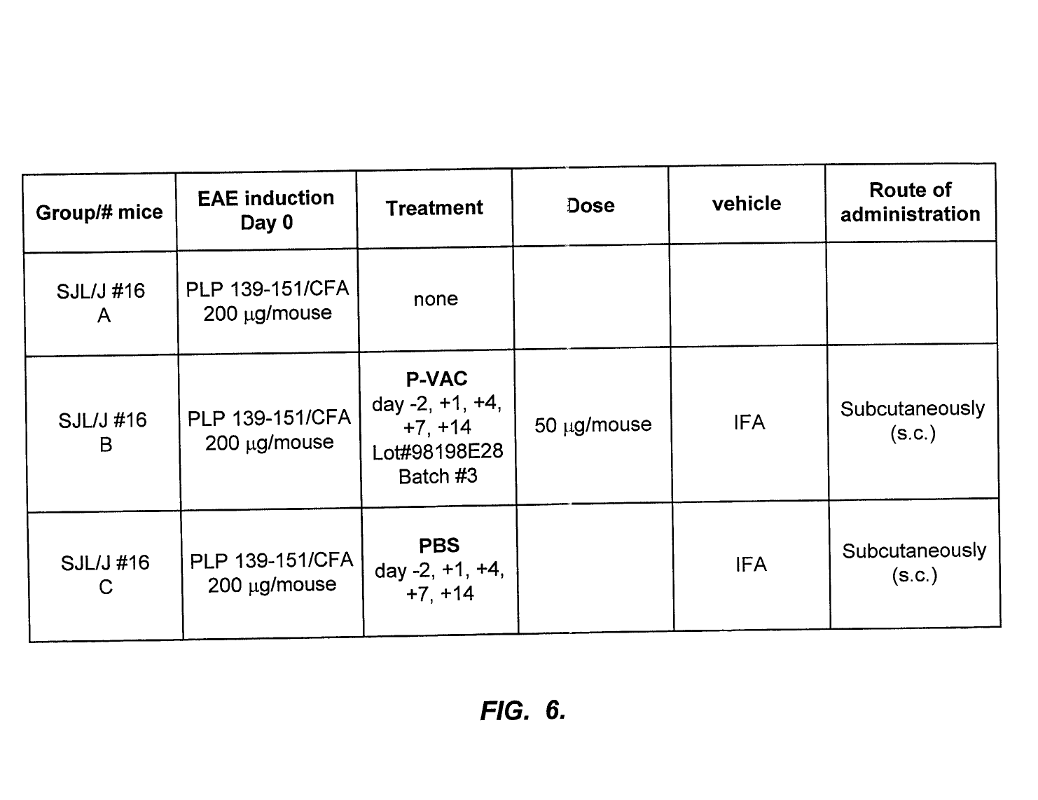

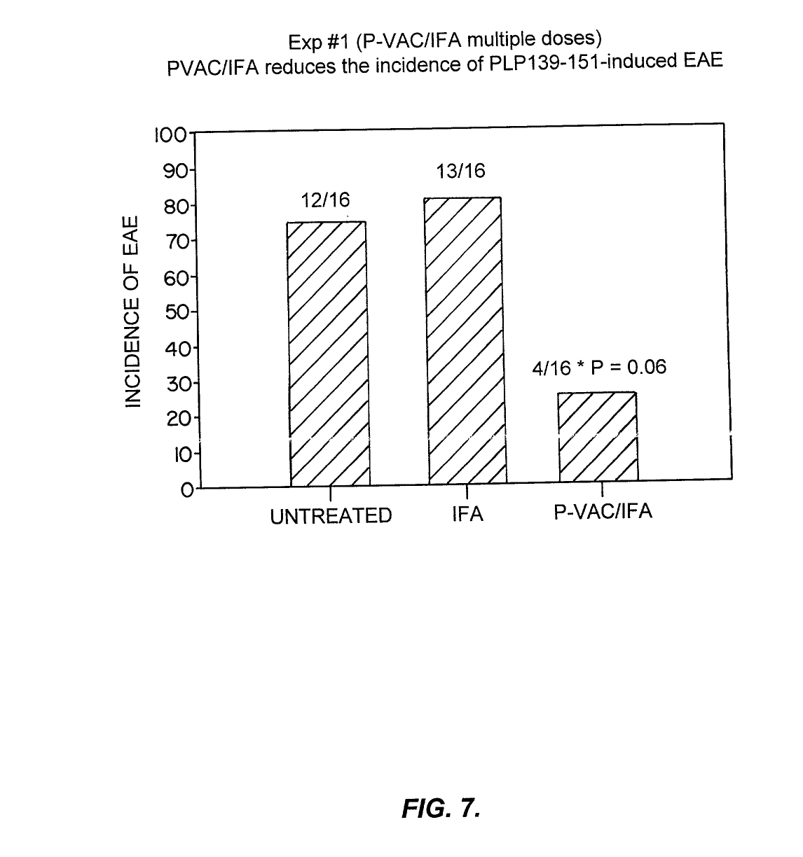

In this experiment, the dose was altered to 50 μg/mouse P-VAC emulsified in IFA. Mice were treated with P-VAC/IFA and injected sc in the flank of the animal at Days −5, −2, 1, 4, 7, and 14 (see FIG. 6). An additional group of mice were treated on the same schedule with IFA alone. Control mice did not receive any treatment (Table 3). Only one significant difference was found in this experiment. Significantly fewer mice in the P-VAC/IFA treatment group displayed disease-related symptoms than did mice in the control group (p=0.033) (Table 4). The results indicate that P-VAC emulsified in IFA has a protective effect when animals are treated both before and after disease induction. These results are also shown in bar graph form in FIG. 7.

TABLE 4

| | | Symptomatic | Fisher's | Paralytic | Fisher's |
|---|---|---|---|---|---|
| Experimental | Sample | Mice | Exact | Mice | Exact |
| Group | Size (n) | Days 1-50 | p-value | Days 1-50 | p-value |
| PVAC/IFA | 16 | 6 | 0.033 | 4 | 0.149 |
| IFA | 16 | 13 | 0.669 | 9 | 1.000 |
| Control | 16 | 12 | | 9 | |

### EXAMPLE 10

### Post Disease Induction Administration of PVAC/IFA can Ameliorate Disease in an EAE Model of Multiple Sclerosis

This example demonstrates the use of delipidated, deglycolipidated M. vaccae to ameliorate disease in experimental allergic encephalomyelitis (EAE), a model for multiple sclerosis. EAE is an autoimmune inflammatory disorder of genetically susceptible mice that is mediated by autoantigen-specific CD4+MHC class II restricted T cells. In susceptible SJL/J mice, the disease can display a relapsing-remitting clinical course of paralysis, which makes it an ideal system to study the efficacy of various immunoregulatory strategies both in the prevention and treatment of disease.

General Protocol

EAE can be induced by using either crude preparations of spinal cord homogenate or myelin or immunodominant encephalitogenic peptides derived from myelin, such as PLP 139-151 peptide. In this case, remitting-relapsing (RR) EAE is induced in female SJL mice (8-12 wks old) by immunization with 200 μg/mouse of the immunodominant peptide PLP 139-151. The peptide is dissolved in PBS and emulsified with an equal volume of CFA containing 600 □g of Mycobacterium tuberculosis H37Ra. Each mouse receives 0.2 ml of the emulsion, which will be administrated subcutaneously (sc) and distributed over three sites on the flank and base of tail. Mice are also given an injection of the coadjuvant pertussis toxin (200 ng/mouse intraperitoneally (ip)) on the day of immunization and 48 hrs later. Beginning approximately 9 days after immunization, animals are weighed daily and observed for the onset of neurological dysfunction. Disease is graded as follows: 0, normal; 0.5, stiff tail; 1, limp tail; 1.5, limp tail with inability to right; 2, paralysis of one limb; 2.5, paralysis of one limb and weakness of one other limb; 3, complete paralysis of both hind limbs; 4, moribund; 5, death. Mice are usually followed for 60-70 days after immunization for relapses.

Protocol and Results for Determining a Minimum Dose of PVAC/IFA that Provides Protection in the EAE Model.

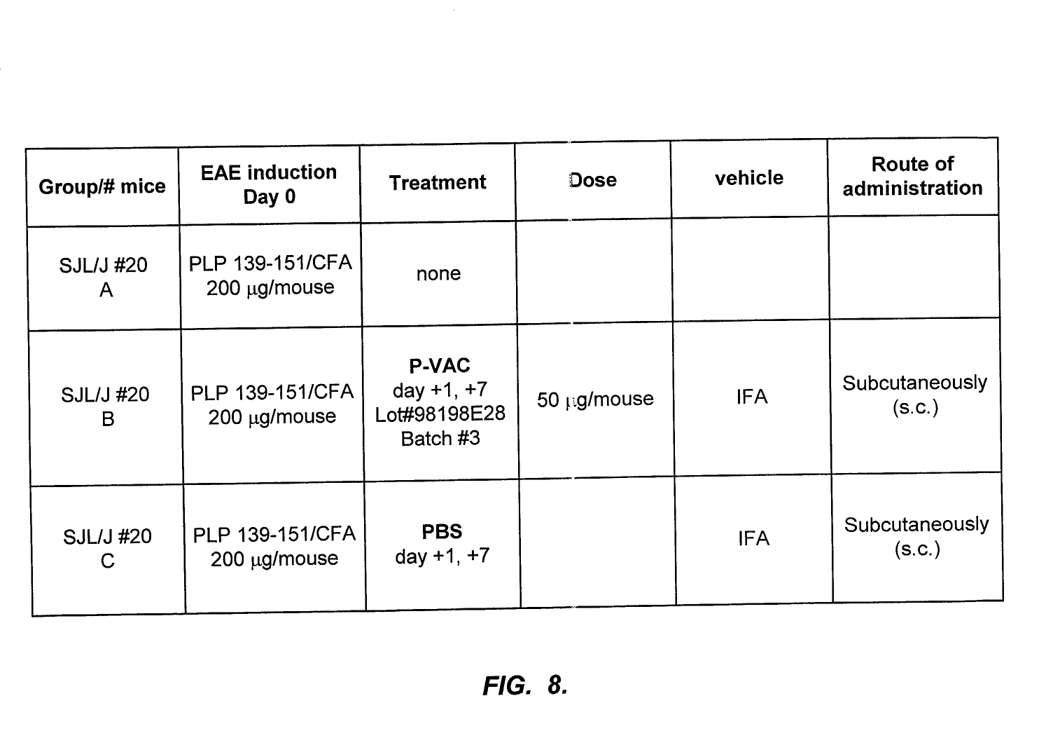

Protocol is outlined in FIG. 8

1. Sixty SJL/J mice were divided into three groups of 20.

2. Disease was induced as described.

3. Group 1 received 50 μg PVAC/IFA administration on day 1 and 7 after disease induction on day 0.

4. Group 2 received IFA administration on day 1 and day 7 after disease induction

5. Group 3 received NO treatment other than disease induction.

6. Mice were evaluated for disease as described above.

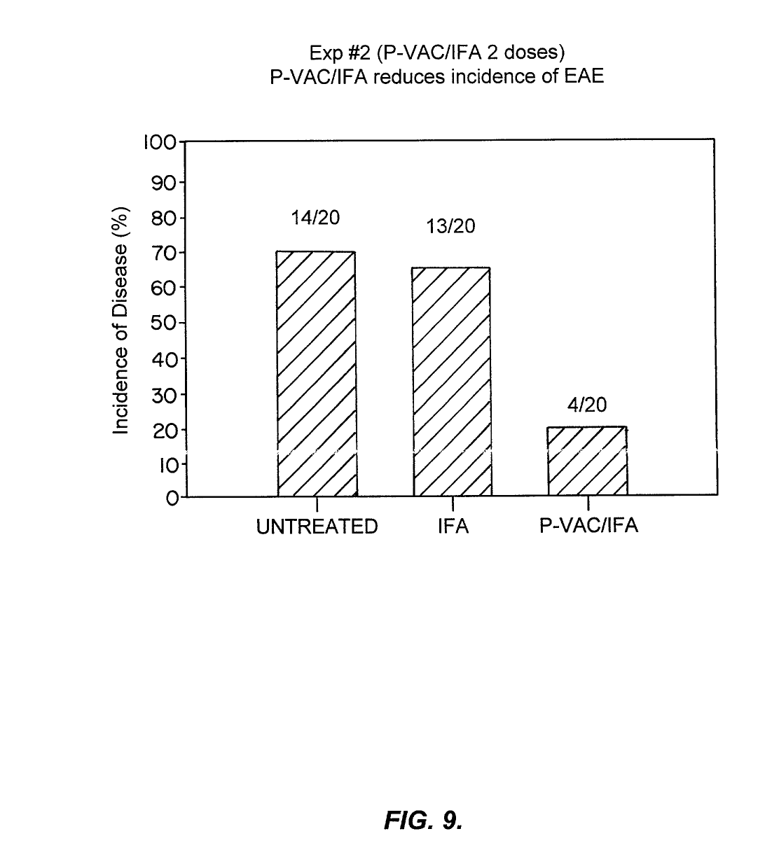

These results demonstrate a protective effect of PVAC/IFA with only 2 injections of 50 □g PVAC emulsified in IFA on two days after disease induction (FIG. 9)

### EXAMPLE 11

### P-VAC/IFA can Act as an Adjuvant for Encephalitogenic PLP Peptide

This example demonstrates the use of delipidated, deglycolipidated M. vaccae cells (P-VAC) as an adjuvant for encephalitogenic PLP peptide.

To better understand the properties of P-VAC as an adjuvant, we have asked the question whether P-VAC alone or in emulsion with IFA could substitute for CFA in delivering the 200 μg encephalitogenic PLP 139-151 peptide and inducing EAE in SJL mice. Peptide+IFA alone is not sufficient to immunize mice and induce EAE.

PLP 139-151 peptide was delivered subcutaneously (sc) to SJL mice (8-12 wks old) in either CFA (control), P-VAC alone, or P-VAC+IFA. Monitoring of disease appearance and statistical analyses were performed as described in Example 5 (page 23, lines 11-21). In this experiment, significantly fewer mice in the group treated with 100 μg P-VAC showed any disease-related symptoms (p<0.001) or signs of paralysis (p=0.005) than did mice in the control group (Table 4). These results demonstrate that P-VAC alone does not substitute for the adjuvant properties of CFA in induction of EAE.

In a second experiment, the protocol was slightly modified. The groups were expanded and PLP 139-151 peptide was delivered sc in either CFA, IFA, P-VAC alone, or P-VAC with IFA. In addition, 35 days after disease induction, animals were rechallenged with PLP 139-151 delivered in CFA.

For statistical analysis of this experiment, the entire set of worst status comparisons was performed twice; once for each of the two experimental phases (i.e. pre- and post-rechallenge at Day 35) (Table 4). In Phase I (Days 1-35), significantly fewer mice treated with IFA displayed disease-related symptoms (p=0.001) or signs of paralysis (p=0.035) than did mice on the control group. Mice treated with 100 μg P-VAC also responded in a similar manner; fewer mice in this group displayed disease-related symptoms (p=0.001) or signs of paralysis than mice in the control group. No significant differences were found between the 100 μg P-VAC/IFA and control groups.

During Phase II of this experiment (after rechallenge at day 35) only one significant difference was noted. More mice treated with 100μγ P-VAC showed signs of paralysis than did mice in the control group (p=0.006) (Table 5).

These results confirm the initial results of Example 8 in that P-VAC alone does not replace the adjuvant activity of CFA in induction of EAE. It appears from these experiments that P-VAC in combination with IFA can substitute for CFA in inducing disease.

Table 4 Analysis of Use PVAC as Adjuvant in EAE Model

The following table (Table 4) summarizes the results of the analyses in a common format. The first column displays labels for the various groups, the second column shows the number of mice (n) in the group, and the third column shows the number of symptomatic mice in that group, i.e., mice with severity grade>0 during the comparison period. The fourth column displays the p-value for the Fisher's exact test comparing the proportion of symptomatic mice in the experimental group vs. the corresponding proportion for the control group. Column 5 of each table shows the number of mice that experienced paralysis or worse (i.e., severity grade≧2) during the comparison period. The sixth column displays the p-value for the Fisher's exact test comparing the proportion of paralyzed mice in the experimental group vs. the corresponding proportion for the control group.

TABLE 5
Analysis of PVAC Effects in EAE Model
Experiment 1

| | | Symptomatic | Fisher's | Paralytic | Fisher's |
|---|---|---|---|---|---|
| Experimental | Sample | Mice | Exact | Mice | Exact |
| Group | Size (n) | Days 1-62 | p-value | Days 1-62 | p-value |
| 100 μg PVAC/ | 12 | 12 | * | 9 | 0.667 |
| IFA | | | | | |
| 100 μg PVAC | 12 | 0 | 0.000 | 0 | 0.005 |
| Control | 12 | 12 | * | 7 | |

Experiment 2 (Phase 1)

| | | Symptomatic | Fisher's | Paralytic | Fisher's |
|---|---|---|---|---|---|
| Experimental | Sample | Mice | Exact | Mice | Exact |
| Group | Size (n) | Days 1-35 | p-value | Days 1-35 | p-value |
| 100 μg PVAC/ | 11 | 6 | 0.659 | 4 | 1.000 |
| IFA | | | | | |
| 100 μg PVAC | 12 | 0 | 0.001 | 0 | 0.029 |
| IFA | 11 | 0 | 0.001 | 0 | 0.035 |
| Control (CFA) | 10 | 7 | | 4 | |

Experiment 2 (Phase 2)

| | | Symptomatic | Fisher's | Paralytic | Fisher's |
|---|---|---|---|---|---|
| Experimental | Sample | Mice | Exact | Mice Days | Exact |
| Group | Size (n) | Days 36-70 | p-value | 36-70 | p-value |
| 100 μg PVAC/ | 11 | 10 | 0.586 | 1 | 0.311 |
| IFA | | | | | |
| 100 μg PVAC | 12 | 12 | 0.195 | 11 | 0.006 |
| IFA | 11 | 7 | 0.635 | 5 | 0.659 |
| Control (CFA) | 10 | 8 | | 3 | |

* No mouse in this group was symptom-free

### EXAMPLE 12

### P-VAC/IFA, but Neither M-VAC nor M-VAC/IFA, can Act as an Adjuvant

This example demonstrates the use of killed M. vaccae cells (M-VAC) and delipidated, deglycolipidated M. vaccae (P-VAC) as adjuvants for encephalitogenic PLP peptide.

To better understand the properties of P-VAC and M-VAC as adjuvants, we have asked the question whether P-VAC or M-VAC alone or in emulsion with IFA could substitute for CFA in delivering the encephalitogenic PLP 139-151 peptide and inducing EAE in SJL mice. Peptide+IFA alone is not sufficient to immunize mice and induce EAE.

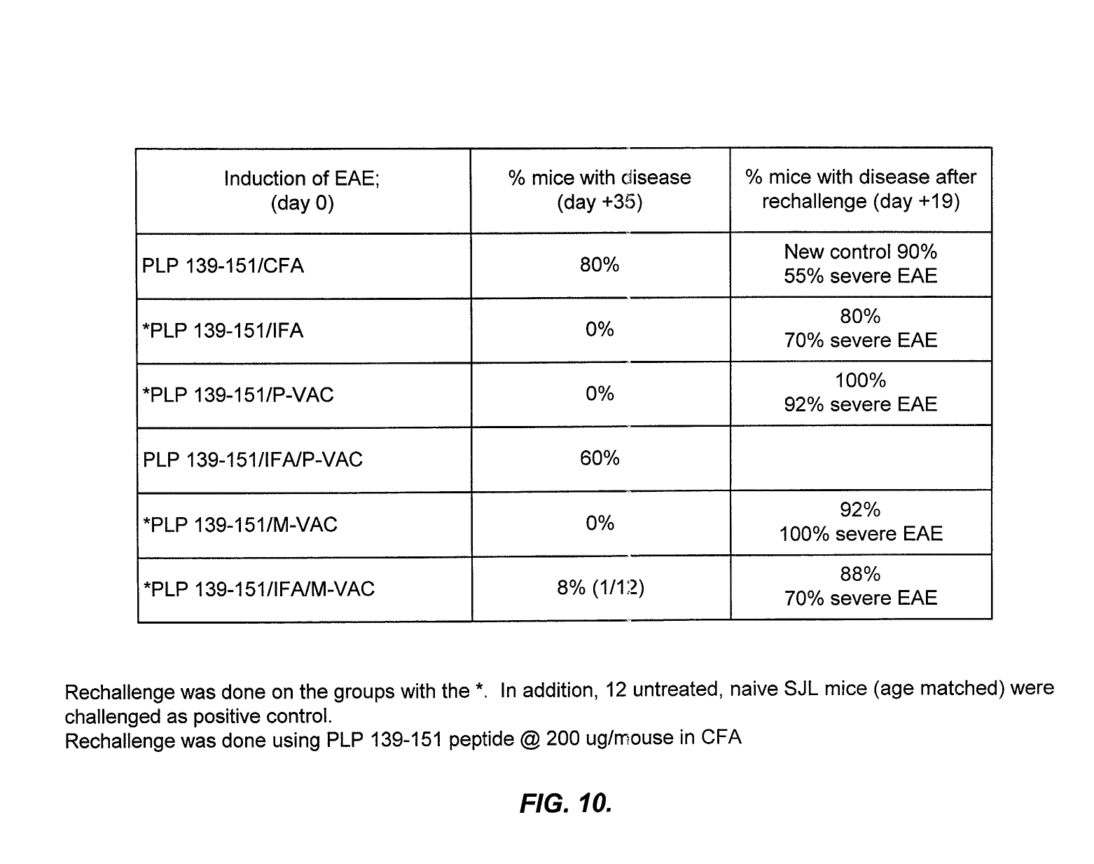

PLP 139-151 peptide was delivered subcutaneously (sc) to SJL mice (8-12 wks old) in either CFA, IFA, P-VAC alone, M-VAC alone, P-VAC+IFA or M-VAC+IFA and animals were monitored for disease appearance and progression. The results are shown in FIG. 10. 35 days after immunization, none of the mice immunized with PLP 139-151 in P-VAC alone showed sign of disease, whereas animal immunized with PLP 139-151 in IFA/P-VAC developed disease (60%), suggesting that IFA+P-VAC can replace the CFA effect in inducing EAE. Interestingly, none of the mice immunized with either M-VAC alone or IFA/M-VAC developed disease, indicating again differences in M-VAC and P-VAC effects.

### EXAMPLE 13

### P-VAC/IFA can Ameliorate Disease in a CIA Model for Human Rheumatoid Arthritis

This example demonstrates the use of killed M. vaccae cells (M-VAC) and delipidated, deglycolipidated M. vaccae (P-VAC) to ameliorate disease in collagen-induced arthritis (CIA) a model for human RA because of 1) observed similarities with respect to synovial inflammation and cartilage/bone destruction; 2) involvement of class II restricted T cell activation in the pathogenesis.

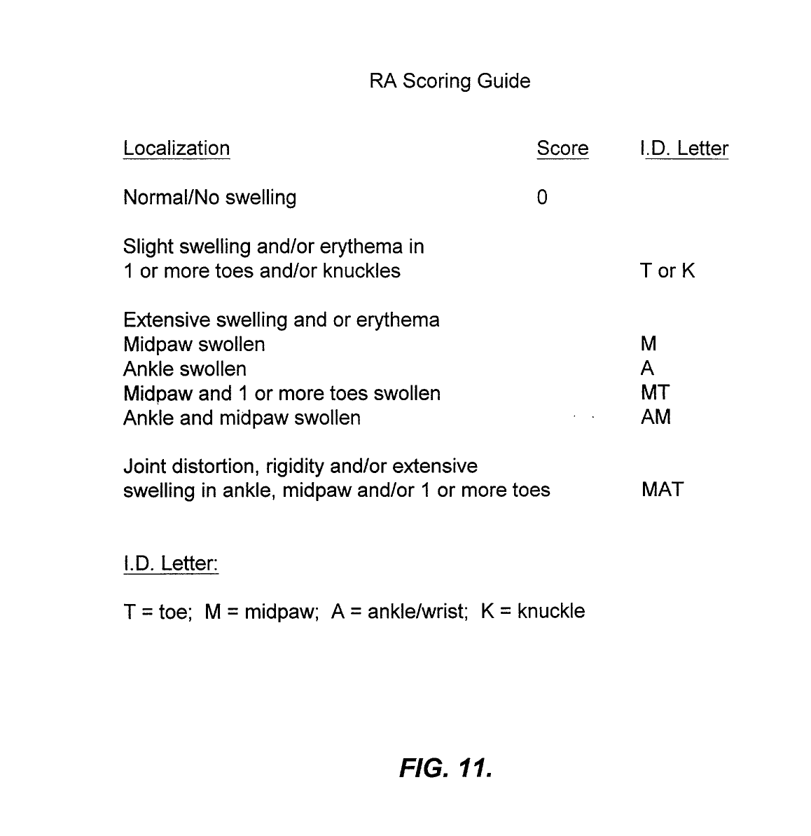

CIA is induced in male DBA/1 mice (8-12 weeks) (10 mice/group) by intradermal (id) injections in the base of the tail (at day=0 and boost at day 21) of chick collagen type II (C11) in CFA. The mean onset is 27-28 days. Animals are assessed for redness and swelling of limbs and clinical score allocated for each mouse>3 times per week. The scoring system is based on the progression of the swelling and/or erythema of the joints up to the stage ofjoint distortion and/or rigidity (see FIG. 11 for RA scoring guide).

Experimental Design and Results

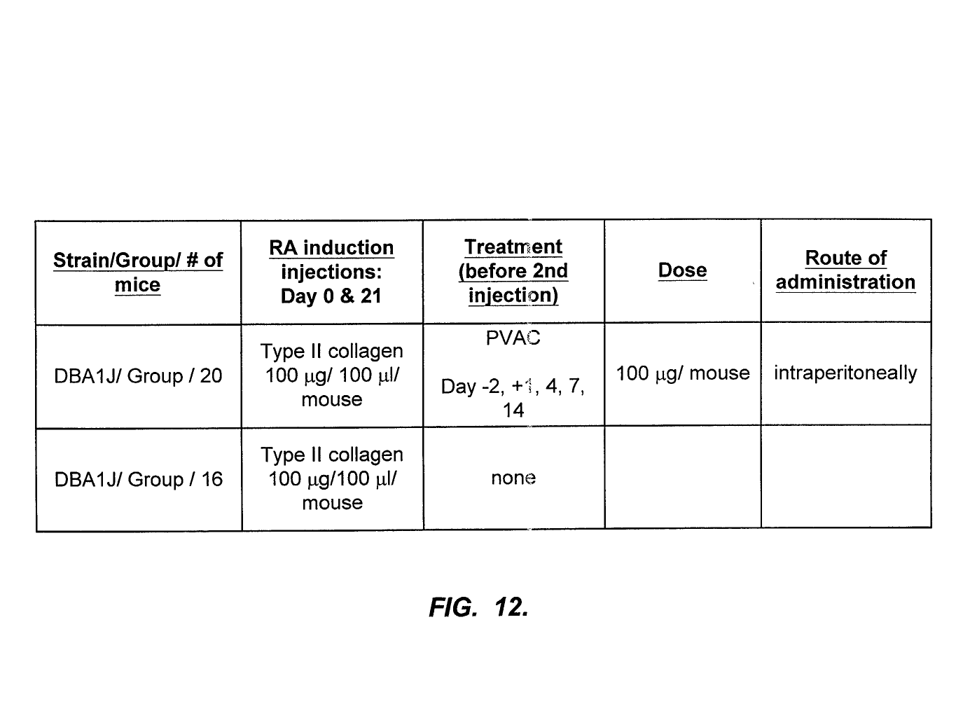

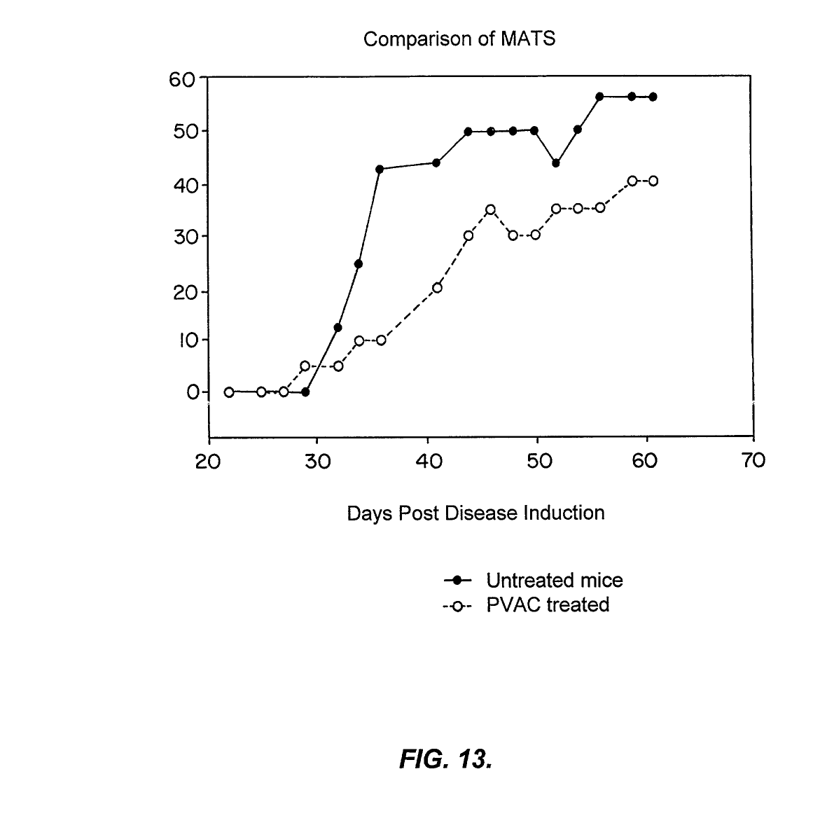

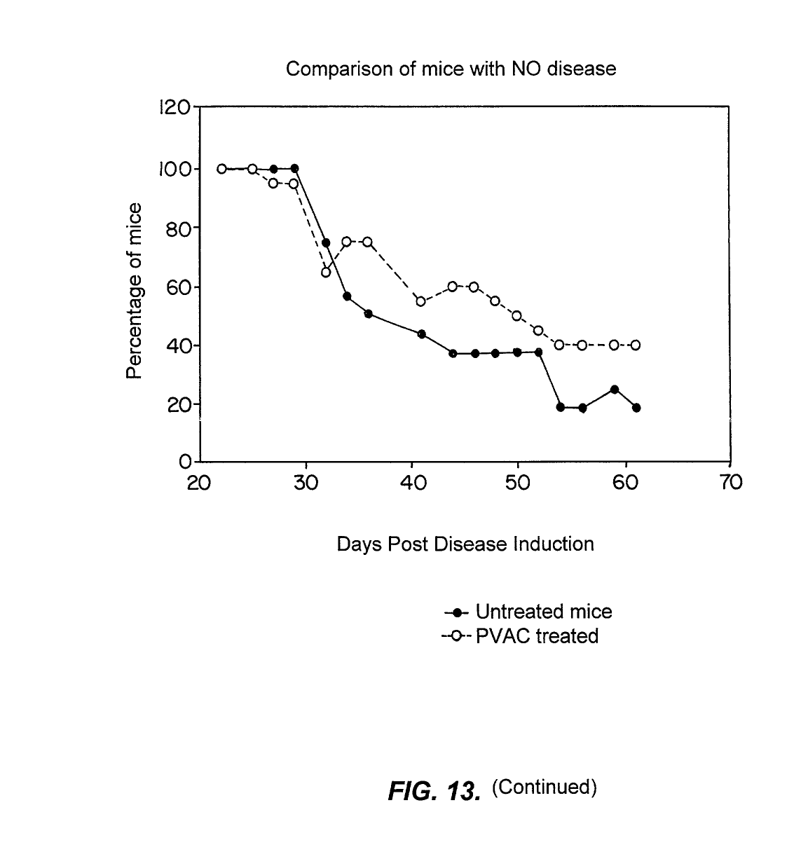

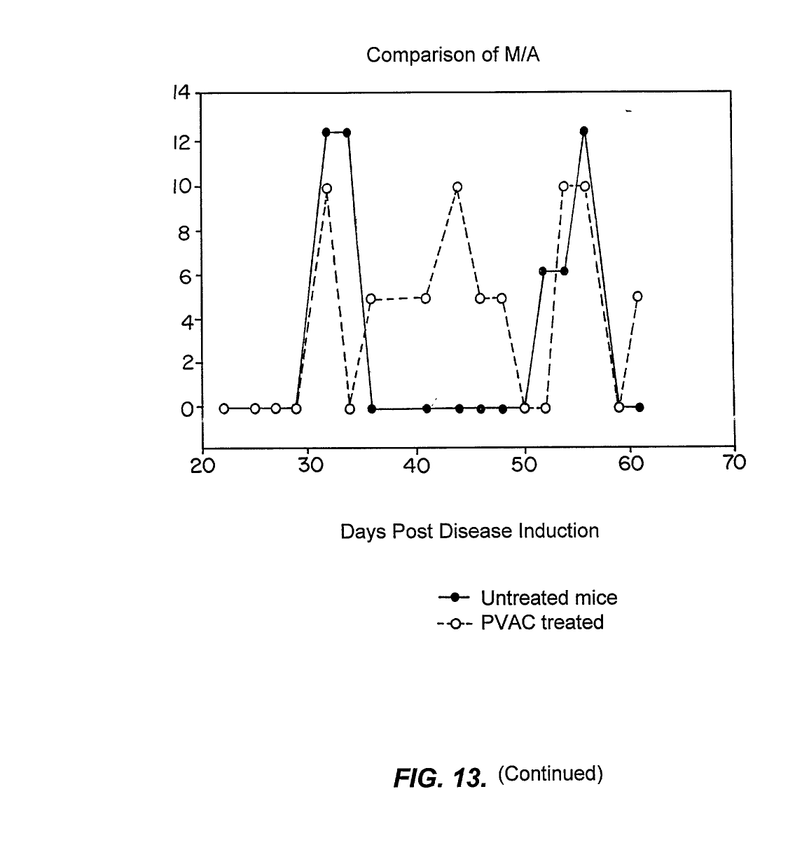

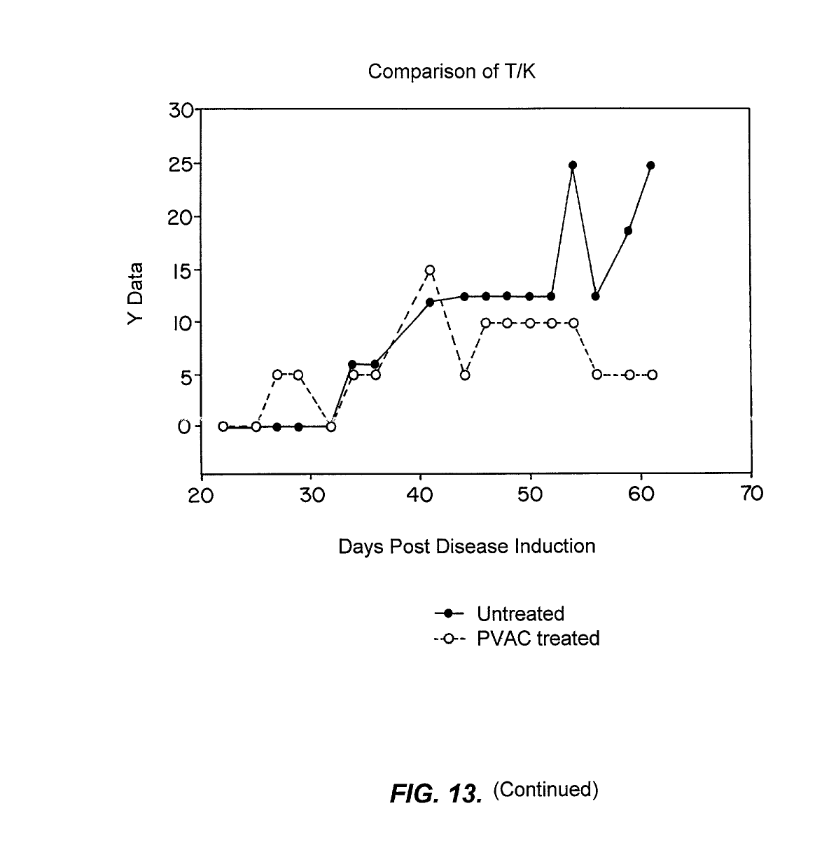

CIA is one of the most widely used polyarthritis model for assessing the role of effects and for determining the efficacy of therapeutic drugs. We have used this protocol to evaluate the effect of P-VAC and M-VAC on this autoimmune disease. Mice received P-VAC (100 □g/mouse intraperitoneally (ip) in PBS) or M-VAC (500 □g/mouse ip in PBS) at the time of the 2^{nd} collagen type II injection according to the protocol described before (e.g. day −2, +1, +4, +7 and +14 after 2^{nd} collagen injection) Experimental protocol is seen in FIG. 12. The results from a first experiment (FIG. 13) indicate a reduction in the number of mice with severe arthritis (defined as MAT) in the group treated with P-VAC compared with the mice treated with M-VAC or untreated control.

### EXAMPLE 14

### P-VAC can Ameliorate Disease in a Mouse Model for Scleroderma

Experiments in two mouse models of scleroderma have suggested that PVAC could have a therapeutic role in this disease. The tight skin mouse (TSK/+) is considered a particularly relevant model for progressive systemic sclerosis in humans. The TSK/+ mouse has a point mutation in the fibrillin 1 gene that leads to dermal thickening and extensive collagen deposition in the skin resembling the skin sclerosis detected in patients with scleroderma. Fibroblasts are abundant, collagen content of the skin is increased, and cultured skin fibroblasts have been found to synthesize almost five times more Type I and Type II procollagen mRNA than fibroblasts from control mice. Additionally, autoantibodies to fibrillin can be detected in both TSK/+ mice and humans with scleroderma however these autoantibodies are thought to be primarily markers for the disease process rather than contributing to the disease process.

Experiments in TSK/+ mouse have demonstrated that PVAC can reverse existing disease in the skin. Ten-month old TSK/+ mice with established disease were treated weekly over the period of two months with subcutaneous injections of PVAC at 1, 10 or 100 μg. Histological evaluation of the skin after the final injection confirmed that PVAC reversed the skin thickness increasingly at doses of 1, 10 and 100 μg compared to saline.

TABLE 6
PVAC Treatment in TSK Mice with Established Disease

| Mice | n | Injected With | Dose (μg) | Skin Thickness (μm) |
|---|---|---|---|---|
| C57bl/6 | 6 | Saline | — | 132 ± 42 |
| TSK | 13 | Saline | — | 216 ± 93 |
| TSK | 5 | PVAC | 1 | 111 ± 14 |
| TSK | 5 | PVAC | 10 | 98 ± 11 |
| TSK | 5 | PVAC | 100 | 83 ± 18 |

To determine if the protective effect could be achieved with fewer doses of PVAC, a second experiment was performed comparing 4, 6 and 8 weekly injections of 100 μg PVAC. Four injections of PVAC reversed existing disease in 2/3 mice while 6 injections were no different from 8 injections in terms of the reversal of established disease. Therefore, in the TSK/+ model, a minimum of 6 injections of PVAC was required to reverse established disease. Additionally, analysis of the sera from these PVAC treated mice demonstrated a dramatic decrease in the anti-fibrillin antibody.

A study was conducted in newborn TSK mice and pa/pa (control mice) in which animals were injected on Day 1 after birth with 100 μg PVAC or on days 1, 7 and 14 after birth. Pa/pa mice were used as controls for this experiment since the fibrillin 1 mutation in TSK mice is linked to a mutation in second gene (Pa) that is known to cause an emphysema-like phenotype in TSK/+ mice. To determine whether PVAC had an effect on this phenotype, the lungs of the mice were also evaluated for emphysema and mice that were wild type at both loci (pa/pa mice) were used as a control Mice were killed at two months of age and skin was harvested and thickness measured.

The skin thickness in the PVAC treated TSK mice was intermediate between the untreated TSK mice and the control group. PVAC treatment had no protective effect on the incidence of emphysema. Results are presented in the table below.

TABLE 7
Prevention of Skin Thickening and Emphysema in TSK/+ vs.
Control Mice

| | | Day of In- | | Skin | # WITH |
|---|---|---|---|---|---|
| | | jection Af- | Compound | Thickness | EMPHY- |
| Mice | n | ter Birth | injected | (μm) | SEMA |
| TSK/+ | 4 | 1 | Saline | 73 ± 14 | 4/4 |
| Pa/pa (control) | 3 | 1 | Saline | 29 ± 3 | 0/3 |
| TSK/+ | 3 | 1 | PVAC (100 ug) | 50 ± 15 | 3/3 |
| TSK/+ | 3 | 1, 7, 14 | PVAC (100 ug) | 52 ± 7 | 3/3 |
| Pa/pa (control) | 3 | 1, 7, 14 | PVAC (100 ug) | 36 ± 15 | 0/3 |

A second model for the dermal thickening observed in human scleroderma is an induced model in which DNA encoding the identical mutated fibrillin gene as found in the spontaneous TSK/+ mouse is administered to normal newborn C57B/6 mice, resulting in the appearance of skin lesions. Consistent with the results in the TSK/+ model, PVAC administered at doses of 10 and 100 μg was able to block the development of dermal thickening in this setting. These results are presented in the table below.

TABLE 8
Prevention of Induced Skin Lesions in Newborn C57B/6 Mice

| Injection | PVAC dose (μg) | n | Skin Thickness (μm) |
|---|---|---|---|
| Saline | 0 | 6 | 33.5 ± 6.7 |
| Plasmid | 0 | 7 | 81.9 ± 11.3 |
| Plasmid | 10 | 6 | 40.4 ± 20.0 |
| Plasmid | 100 | 4 | 49.5 ± 9.8 |

Together these results show that PVAC not only blocks the development of dermal thickening but also reverses established disease in the skin.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. A method for the treatment of scleroderma, the method comprising administering an immunogenically effective dose of a pharmaceutical composition comprising P-VAC..

2. The method of claim 1, wherein the P-VAC is administered with an immunostimulant.

3. The method of claim 2, wherein the immunostimulant is an adjuvant selected from the group consisting of mineral oil, Bortadella pertussis, Freund's Incomplete Adjuvant, Freund's Complete Adjuvant, CWS, TDM, Leif, an aluminum salt, GM-CSF, interleukin-2, interleukin-7, interleukin-12, Montanide ISA 720, SAF, ISCOMS, MF-59, an SBAS adjuvant, and Detox.

4. The method of claim 2, wherein the immunostimulant is an adjuvant selected from the group consisting of AS-2 or derivative thereof, a biodegradable microsphere, monophosphoryl lipid A (MPL), 3-de-O-acylated monophosphoryl lipid A (3D-MPL), Quil A, CpG, a saponin or mimetic thereof, and an aminoalkyl glucosaminide 4-phosphate.

5. The method of claim 1, wherein the P-VAC is administered parenterally.

6. The method of claim 5, wherein the P-VAC is administered subcutaneously.

7. The method of claim 5, wherein P-VAC is administered intraperitonealy.
